(19) United States
(12) Patent Application Publication
(10) Pub. No.: US 2025/0132038 A1
(43) Pub. Date: Apr. 24, 2025

(54) AI-BASED CONTENT GENERATION AND VERIFICATION

(71) Applicant: Healthcare Competencies, LLC, Liberty Lake, WA (US)

(72) Inventor: Grant Ackley ELY, Liberty Lake, WA (US)

(21) Appl. No.: 18/923,636

(22) Filed: Oct. 22, 2024

Related U.S. Application Data
(60) Provisional application No. 63/592,331, filed on Oct. 23, 2023.
(60) Provisional application No. 63/553,999, filed on Feb. 15, 2024.

(51) Int. Cl. G16H 50/20; G16H 10/60; G16H 50/50

(52) U.S. Cl. CPC G16H 50/20; G16H 10/60; G16H 50/50

(57) ABSTRACT

Techniques relate to generating and/or verifying content based on protocols and/or user input. The techniques can receive protocol data from an entity, including a procedure, guideline, policy, or standard. User input requesting content generation related to specific parameters can also be received. Based on the protocol data, a data request can be generated and sent to a content generation component. Machine-generated content can be received from the content generation component in response. The machine-generated content can be analyzed based on the protocol data. Output content can be generated based on the machine-generated content and provided to a computing device. In examples, this ensures that generated content aligns with entity-specific protocols and/or user-specified parameters, enabling tailored and/or compliant content delivery for various contexts.

### CROSS-REFERENCE TO RELATED APPLICATIONS

This application claims priority to U.S. Provisional Application No. 63/592,331, filed Oct. 23, 2023, and U.S. Provisional Application No. 63/553,999, filed Feb. 15, 2024, the entire contents of which are incorporated herein by reference.

### BACKGROUND

In conventional Artificial Intelligence (AI) systems, the generation of inaccurate or erroneous information may occur due to various factors such as incomplete training data, algorithmic limitations, or computational constraints. Such inaccuracies may lead to detrimental outcomes in certain application domains. For instance, in healthcare-related implementations, the propagation of incorrect information may potentially result in severe adverse effects, including but not limited to improper training, improper diagnosis, inappropriate treatment recommendations, or suboptimal patient care protocols. Furthermore, numerous existing AI systems may suffer from temporal inconsistencies in their knowledge bases, potentially disseminating outdated or obsolete information when utilized for educational or informational purposes across diverse subject matters.

### BRIEF DESCRIPTION OF THE DRAWINGS

Various examples are depicted in the accompanying drawings for illustrative purposes and should not be interpreted as limiting the scope of the disclosure. In addition, various features of different disclosed examples can be combined to form additional examples, which are part of this disclosure. Throughout the drawings, reference numbers may be reused to indicate correspondence between reference elements.

FIG. 1 illustrates an example architecture in which the techniques described herein may be implemented.

FIG. 2 illustrates additional example details of the service provider of FIG. 1 according to one or more examples.

FIG. 3 illustrates details of an example user device according to one or more examples.

FIG. 4A and 4B illustrate a flowchart of an example process for generating and verifying machine-generated content according to one or more examples.

FIGS. 5A, 5B, and 5C illustrate example user interfaces for facilitating vocabulary mastery and training according to one or more examples.

FIGS. 6A, 6B, and 6C illustrate example user interfaces for facilitating clinical training according to one or more examples.

FIGS. 7A, 7B, and 7C illustrate example user interfaces for training a user on lab tests according to one or more examples.

FIG. 8 illustrates an example user interface for training a user on medications according to one or more examples.

### DETAILED DESCRIPTION

In examples, techniques and architectures discussed herein relate to a platform that provides functionality to create machine-generated content and/or verify the accuracy of the content. For example, content can be generated based on a request that indicates one or more data constraints. Once generated, the content can be analyzed to ensure that the content complies/conforms with one or more criteria to ensure accuracy, consistency, and/or conformity. The content can be output to a user, system, component, etc. This process can improve the quality of machine-generated content and/or enhance the efficiency of content generation. In some cases, machine-generated content can be updated, such as when the machine-generated content does not satisfy one or more criteria. This can further ensure that content that is provided to a system, user, or others is accurate.

Although certain examples are disclosed below, the subject matter extends beyond the specifically disclosed examples to other alternative examples and/or uses, and to modifications and equivalents thereof. Thus, the scope of the claims that may arise here from is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein. Although various examples are discussed in the medical context, the techniques and architectures can be implemented in other contexts.

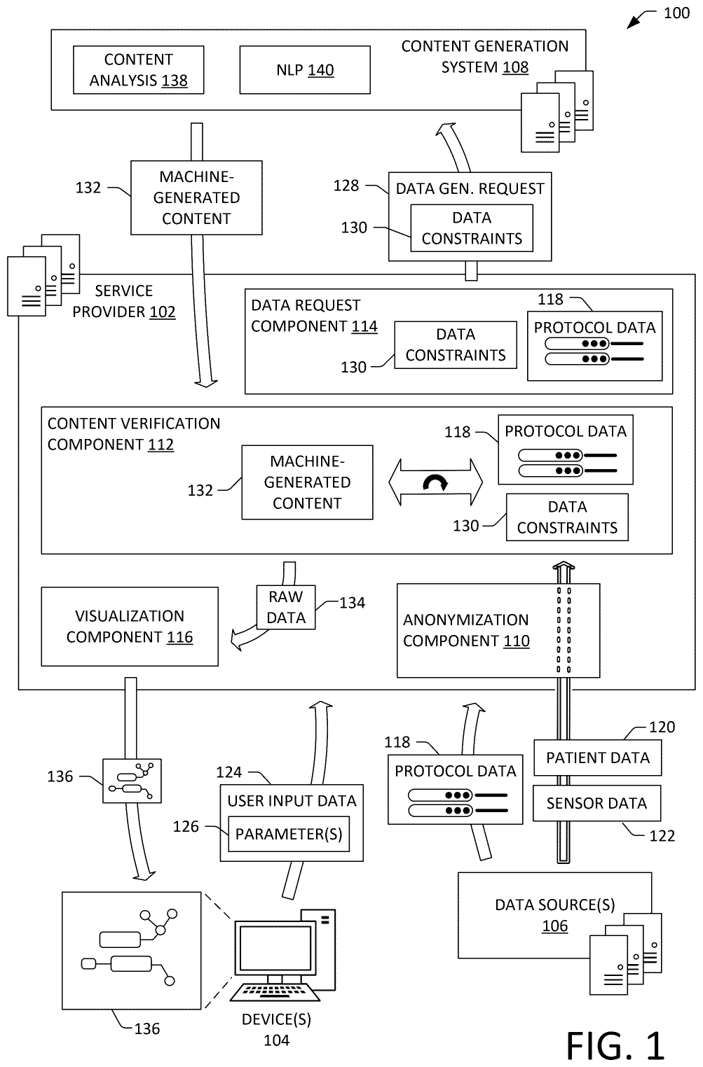

FIG. 1 illustrates an example architecture 100 in which the techniques described herein can be implemented. The architecture 100 includes a service provider 102 configured to manage generation of content, verify the accuracy of generated content, distribute the generated content, and/or perform a variety of other operations. The architecture 100 can include one or more devices 104, data source 106, and/or a content generation system 108 that are each configured to communicate with the service provider 102 and/or each other over one or more networks. In examples, one or more of the service provider 102, one or more devices 104 (hereinafter “the device 104”), data source 106 (hereinafter “the data source 106”), and/or content generation system 108 (also referred to as “the content generation component 108”) can be associated with a user. Although the service provider 102, content generation system 108, and data source 106 are illustrated as separate components, one or more elements of the service provider 102, content generation system 108, and data source 106 can be combined. For example, the data source 106 and/or content generation system 108 can be implemented as part of the service provider 102.

The service provider 102 can include an anonymization component 110, content verification component 112, data request component 114, and/or visualization component 116 to facilitate various functionality discussed herein. For example, the anonymization component 110 can be configured to anonymize data, the content verification component 112 can be configured to manage the creation of content and/or analyze machine-generated content, the data request component 114 can be configured to generate data requests to cause data to be generated that align with certain criteria, the visualization component 116 can be configured to provide visual content and/or interface with the device 104. The anonymization component 110, content verification component 112, data request component 114, and/or visualization component 116 can be configured to operate in cooperation and/or exchange data (e.g., communicate) to facilitate operations.

The anonymization component 110 can be configured to anonymize data by removing user identification information from the data. For example, the data source 106 can provide protocol data 118, patient data 120, sensor data 122, and/or other data to the service provider 102, wherein the anonymization component 110 can be configured to remove user identification data and/or store anonymized data at the service provider 102.

Example anonymization processes can include:
Removing or encrypting direct identifiers such as names, social security numbers, medical record numbers, etc.
Generalizing quasi-identifiers like dates of birth to broader age ranges or categories.
Aggregating sensitive data to higher levels, such as reporting diagnoses by general categories rather than specific conditions.
Applying data perturbation techniques to add controlled noise to numerical values while preserving overall statistical properties.
Implementing k-anonymity to ensure each record is indistinguishable from at least k−1 other records for certain identifying attributes.
Using pseudonymization to replace identifiable information with artificial identifiers that cannot be traced back to individuals without additional information.
Suppressing or redacting certain fields or entries that may lead to identification when combined with other data.
Employing data masking to replace sensitive information with realistic but fictitious data.
Applying differential privacy techniques to add statistical noise to query results, protecting individual privacy while allowing meaningful analysis.
Utilizing synthetic data generation to create artificial records that maintain the statistical properties of the original dataset without containing actual patient information.

In examples, patient data can include electronic health records and/or data from electronic health records. Patient data can encompass a wide range of information, including:
Demographic information like age, gender, ethnicity, and address.
Medical history, including past diagnoses, surgeries, and treatments.
Current health conditions and/or ongoing treatments.
Medication lists, including dosages and/or frequencies.
Allergies and/or adverse reactions to medications or treatments.
Laboratory test results and/or diagnostic imaging reports.
Vital signs and/or other physiological measurements.
Immunization records.
Family medical history.
Lifestyle factors such as smoking status, alcohol consumption, and/or exercise habits.
Mental health assessments and/or psychiatric evaluations.
Genetic test results and/or genomic data.
Social determinants of health, including occupation and/or living conditions.
Insurance and/or billing information.
Appointment history and/or scheduled future appointments.
Notes from healthcare providers, including observations and/or treatment plans.
Emergency contact information.
Advance directives and/or end-of-life care preferences.
Telemedicine visit records and/or remote monitoring data.
Wearable device data, such as heart rate and/or activity levels.

In examples, sensor data includes data generated by a medical system/device. Sensor data can encompass a wide range of data, including:
Vital sign measurements from patient monitors, such as heart rate, blood pressure, respiratory rate, and/or oxygen saturation levels.
Continuous glucose monitoring data from diabetic patients.
Electrocardiogram (ECG) readings from cardiac monitoring devices.
Electroencephalogram (EEG) data from brain activity monitors.
Temperature readings from smart thermometers or thermal cameras.
Infusion pump data, including medication flow rates and/or total volume administered.
Ventilator data, such as tidal volume, respiratory rate, and/or oxygen concentration.
Sleep study data from polysomnography devices.
Fetal heart rate and/or contraction data from maternal-fetal monitors.
Intracranial pressure measurements from neurosurgical monitoring devices.
Pulse oximetry data for continuous oxygen saturation monitoring.
Capnography readings for end-tidal CO2 monitoring.
Bioimpedance measurements for fluid status assessment.
Accelerometer data from fall detection devices.
Weight measurements from smart hospital beds.
Urine output data from catheter sensors.
Blood gas analyzer results for arterial blood samples.
Spirometry data for lung function assessment.
Telemetry data from implantable cardiac devices.
Pressure mapping data from wound care devices.

Although the anonymization component 110 is illustrated as part of the service provider 102, the anonymization component 110 can be implemented by the data source 106, content generation system 108, device 104, and/or other devices/systems. In examples, anonymized data is sent to the service provider 102. That is, data can be anonymized before it is sent to the service provider 102.

As noted above, the service provider 102 can receive protocol data 118 from the data source 106. The protocol data 118 can include/indicate procedures, guidelines, policies, or standards defined by an entity, such as an organization. Example protocol data can include/indicate:
Clinical protocols (e.g., step-by-step procedures for treating specific conditions or performing certain medical interventions).
Care pathways (e.g., standardized, evidence-based multidisciplinary management plans for specific conditions or patient populations).
Standard operating procedures (SOPs) (e.g., detailed instructions for routine operational tasks or processes).
Medication administration guidelines (e.g., specific rules and/or procedures for administering medications within the hospital).
Infection control policies (e.g., procedures for preventing and managing infections within the hospital environment).
Emergency response protocols (e.g., procedures for handling various types of medical emergencies or disasters).
Patient assessment tools (e.g., standardized methods for evaluating patient conditions or risk factors).
Discharge planning procedures (e.g., guidelines for preparing patients for discharge and/or follow-up care).
Interdepartmental communication protocols (e.g., procedures for sharing information between different hospital units or specialties).
Equipment usage guidelines (e.g., instructions for proper use and maintenance of medical devices and/or equipment).
Documentation standards (e.g., specific requirements for recording patient information and/or clinical activities).
Ethical guidelines (e.g., procedures for handling ethical dilemmas or making difficult clinical decisions).
Quality improvement initiatives (e.g., specific programs or procedures aimed at enhancing patient care quality).
Staff training protocols (e.g., standardized methods for onboarding new staff or providing continuing education).
Patient education materials (e.g., hospital-specific information provided to patients about their conditions or treatments).
Referral procedures (e.g., guidelines for referring patients to specialists or other healthcare facilities).
Telemedicine protocols (e.g., procedures for conducting remote consultations or monitoring).
Nutritional guidelines (e.g., hospital-specific dietary recommendations for various patient conditions).
Visitor policies (e.g., rules and/or procedures for managing patient visitors, which may vary by department or patient condition).
Clinical workflow algorithms.
Standardized care protocols.
Clinical process models.
Evidence-based intervention frameworks.
Structured clinical pathways.
Computerized clinical guidelines.
Clinical decision trees.
Standardized order sets.
Clinical practice optimization models.
Integrated care protocols.

In some instances, protocol data is based on (or includes) medical content items, such as journal articles, medical textbooks, reference guides, clinical practice guidelines, case reports and/or case series, systematic reviews and/or meta-analyses, medical conference proceedings and/or presentations, clinical trial protocols and/or results, medical imaging studies and/or reports, medical education materials and/or curricula, patient education brochures and/or pamphlets, medical podcasts and/or webinars, healthcare policy documents and/or regulations, medical news articles and/or press releases, medical encyclopedias and/or dictionaries, anatomical atlases and/or diagrams, drug monographs and/or pharmacological references, medical device manuals and/or technical specifications, public health reports and/or epidemiological studies, medical ethics guidelines and/or position papers, medical simulation scenarios and/or training materials, medical quality improvement reports, healthcare technology assessment reports, medical laboratory procedure manuals, etc.

In some examples, protocol data is specific to a particular context/entity (sometimes referred to as “entity-specific protocol), such as specific to a hospital, department within a hospital, organization, group of individuals, type of employee, job title, geographical region (e.g., northwest has different protocol data than the southwest), etc. For example, the service provider 102 can be configured to provide tailored content for a specific hospital, wherein the hospital can upload protocol data that has been created by the hospital for treating certain situations (e.g. medical conditions). In other words, the hospital may have its own way of handling certain situations and the service provider 102 can customize functionality (e.g., content generation) to the specific hospital. To illustrate, the service provider 102 can cause training data to be generated that is customized to protocol data that is specific to a hospital. This can allow the hospital to train medical professionals on the specific protocols of the hospital. In some cases, machine-generated content provides an indication as to content that is specific to protocol data, such as by highlighting, bolding, italicizing, underlining, coloring, etc. content that within machine-generated content that is aligned with protocol data. This can allow a user to view what is specific to a certain context, such as aligned with protocol from a hospital.

In some examples, protocol data is based on (or includes) voting data provided by one or more users. For instance, a group of users (which may be associated with certain permissions, such as a select group of users) can vote on training data, protocols, medical conditions, or any type of information that may be relevant for training new medical professionals. Here, protocol data can be generated that indicates the items/information that received the greatest number of votes. In examples, such protocol data (that is based on voting) can be used to generate machine-generated content, such as to generate content that is most aligned with the top voted item/information.

The data request component 114 can be configured to process the protocol data 118, user input data 124 (which can include one or more parameters 126), patient data 120, sensor data 122, and/or other data to generate a data generation request 128. The data generation request 128 can include one or more constraints 130 for generating content, such as limitations for the content generation system 108 to generate content. The data generation request 128 can request that content be generated by the content generation system 108. In examples, the data generation request 128 requests that certain types of content be generated by the content generation system 108. The data generation component 114 can communicate with the content generation system 108, such as by sending the data generation request 128 to the content generation system 108.

The data generation request 128 can include/indicate one or more data constraints 130 (also referred to as “limitations” or “data generation constraints”) for generating content, such as limitations for an AI model to generate content. In examples, the one or more data constraints 130 are based on (or include) the user input data 124, one or more parameters 126, protocol data 118, patient data 120, sensor data 122, and/or analytics data,

Example data constraints can include:
Topic restrictions to ensure generated content remains within specified subject areas.
Vocabulary limitations to control the complexity or specificity of language used.
Output length parameters to define the desired size of generated content.
Tone and/or style guidelines to maintain consistency with organizational voice.
Factual accuracy requirements based on verified data sources.
Time period limitations to focus on current or historical information.
Demographic considerations to tailor content for specific audience groups.
Ethical and/or legal boundaries to prevent generation of inappropriate or non-compliant content.
Format specifications to structure the output in a particular way.
Source citation requirements to ensure traceability of information.
Confidentiality filters to exclude sensitive or protected information.
Geographic relevance parameters to focus on region-specific content.
Technical terminology usage guidelines.
Readability level targets to ensure accessibility for intended audiences.
Multilingual constraints for generating content in specific languages.
Contextual relevance parameters to align with specific use cases or scenarios.
Bias mitigation rules to promote fairness and/or inclusivity.
Temporal constraints to generate content relevant to specific time frames.
Regulatory/protocol compliance checks to adhere to industry-specific standards/protocols.
Personalization factors to tailor content to individual user profiles or preferences.

In some instances, the data generation request 128 can request that content be generated based on the protocol data 118 and/or additional content be generated/added based on the knowledge base/data source of the content generation system 108 (or another knowledge base/data source). For example, the data generation request 128 can include a priority/preference for generating content from content in the protocol data 118. Here, the data generation request 128 can include the protocol data 118 and/or the protocol data 118 can be represented in the data constraints 130. The content generation system 108 can first determine if the protocol data 118 (and/or other portions of the data generation request 128) provides sufficient content/guidance (e.g., satisfies one or more criteria) for generating content. If so, the machine-generated content 132 can generate content (for the machine-generated content 132) from the protocol data 118 and/or generate further content (for the machine-generated content 132) from other sources to fill in the gaps of the protocol data 118, if needed.

The content verification component 112 can be configured to analyze machine-generated content 132 received back from the content generation system 108 in response to sending the data generation request 128. For example, the content verification component 112 can be configured to determine if the machine-generated content 132 satisfies one or more criteria. In some instances, the content verification component 112 can analyze the machine-generated content 132 in relation to the protocol data 118, user input data 124, parameter(s) 126, data constraints 130, and/or data generation request 128 to evaluate/determine if the machine-generated content 132 satisfies one or more criteria (e.g., align with one or more of such data). In some instances where the machine-generated content 132 does not satisfy one or more criteria, the content verification component 112 can request that updated content be generated. The content verification component 112 can communicate with the data request component 114 to cause the data request component 114 to place additional data constraints on a data request (e.g., add more limiting, specific, etc. constraints). Further, in some instances where the machine-generated content 132 does not satisfy one or more criteria, the content verification component 112 can update the machine-generated content 132 to more closely align with the one or more criteria.

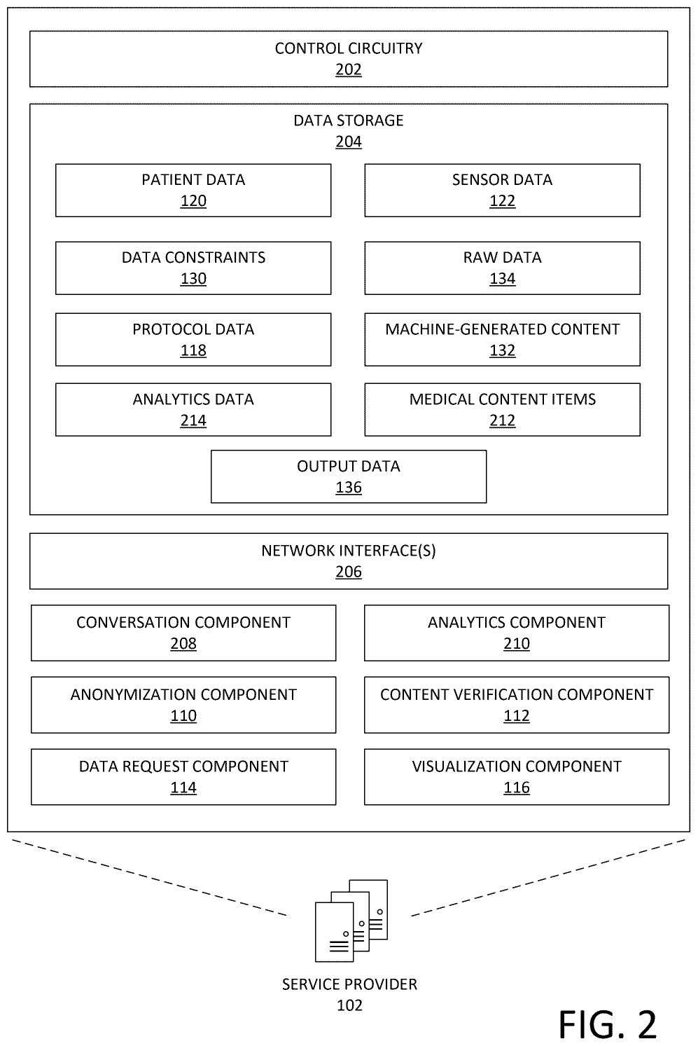

In examples, the content verification component 112 can analyze the machine-generated content 132 relative to analytics data indicating trends in medical conditions. The analytics data can be determined by analyzing data, as discussed in further detail below in relation to FIG. 2. In some instances, the content verification component 112 can reorder/reorder/rank/update the machine-generated content 132 such that more recent trends in medical conditions are presented more predominantly (e.g., ranks higher, emphasized, etc.) in raw data 134 that is provided to the visualization component 116.

The visualization component 116 can be configured to generate output data 136 (e.g., graphical data, audio data, haptic data, etc.) to be presented within an interface. For example, the visualization component 116 can receive/obtain raw data 134 from the content verification component 112, such as raw data generated by the content generation system 108 and/or verified by the content verification component 112. In examples, the raw data 134 is the machine-generated content 132 received from the content generation system 108 with or without being modified by the content verification component 112. In examples, the output data 136 (also referred to as “output content,” “displayable content,” or “user interface content”) can represent a user interface, one or more visual representations for elements of the user interface, and so on. The output data 136 can be provided to the device 104 and/or another device/component to view/interact with data generated by the content generation system 108. In some examples, the visualization component 110 can enable raw data and/or other forms of data to be visualized in an efficient manner to understand complex or unintelligible machine-generated content.

As noted above, the content generation system 108 can generate content 132 (also referred to as “machine-generated content 132”). For example, in response to receiving the data generation request 128, the content generation system 108 can generate the machine-generated content 132 based on the data constraints 130, such as by generating content that aligns with/satisfies the data constraints 130.

Machine-generated content can include a wide variety of data types including:
Textual content, such as responses, articles, reports, narratives, summaries, patient education materials, research abstracts, detailed medical protocols, question-answer pairs for educational/training, rationale/reasoning for a correct/incorrect answer, simulated patient case studies, treatment plans, recommendations, etc.
Image-based data, such as still images (e.g., synthetic medical illustrations, anatomical diagrams, visual representations of statistical data, etc.), video images (e.g., animated explanations of medical procedures, simulated patient interactions, visual guides for rehabilitation exercises, etc.), medical imaging data (e.g., synthetic/original X-rays, MRIs (Magnetic Resonance Imaging), or CT (Computed Tomography) scans for training or demonstration purposes), graphs, charts, etc.
Audio data, such as synthesized speech for text-to-speech applications, simulated patient conversations for training purposes, generated ambient sounds for virtual reality medical simulations, audio descriptions of medical procedures, voice-overs for educational content, audio-based quizzes/assessments, etc.
3-Dimensional (3D) data, such as 3D models of anatomical structures, virtual reality environments for surgical training, interactive 3D visualizations of complex molecular structures, 3D representations of medical devices, virtual patients for simulation purposes, produce 3D-printable models for educational or pre-surgical planning applications, etc.
Structured data, such as synthetic electronic health records, simulated laboratory results, medication lists, time-series data simulating patient vital signs or disease progression, decision trees for clinical decision support systems, synthetic datasets for machine learning model training in healthcare applications, etc.

In examples, the content generation system 108 can implement one or more Artificial Intelligence (AI) models configured to generate content. For instance, the content generation system 108 can implement a large language model (LLM) utilizing a transformer-based neural network architecture. The LLM can be pre-trained on a diverse corpus of text data, including but not limited to medical literature, clinical guidelines, healthcare protocols, etc. The LLM can employ self-attention mechanisms and/or multiple transformer layers to capture complex relationships within the input data and/or generate contextually relevant content. In some implementations, the LLM can utilize techniques such as few-shot learning or in-context learning to adapt to specific domains or tasks without extensive fine-tuning.

The content generation system 108 can incorporate various types of LLMs, each with distinct characteristics and capabilities. For instance, autoregressive models like GPT (Generative Pre-trained Transformer) can generate text sequentially, predicting each token based on the previous tokens. In contrast, masked language models such as BERT (Bidirectional Encoder Representations from Transformers) can understand context from both directions, making them particularly effective for tasks like text classification and/or named entity recognition. Encoder-decoder models, exemplified by T5 (Text-to-Text Transfer Transformer), can be versatile in handling multiple natural language processing tasks within a unified framework.

In addition to LLMs, the content generation system 108 can utilize other AI-based technologies and/or approaches. These can include rule-based expert systems that leverage predefined knowledge bases and inference engines to generate content based on specific rules and/or heuristics. Natural Language Generation (NLG) systems, which can convert structured data into human-readable text, can be employed for tasks such as generating clinical reports or summarizing patient data. Retrieval-based systems can generate content by selecting and combining relevant information from pre-existing databases or document collections. Furthermore, hybrid approaches that combine multiple techniques, such as neural-symbolic systems integrating neural networks with symbolic reasoning, can be implemented to leverage the strengths of different AI paradigms. In some implementations, the content generation system 108 can also incorporate generative adversarial networks (GANs) or variational autoencoders (VAEs) for tasks involving the generation of synthetic data or creative content.

In examples, the content generation system 108 can implement more specialized LLMs tailored for healthcare applications. These models can be fine-tuned on domain-specific datasets to enhance their performance in generating medically accurate and relevant content. Some implementations can utilize multi-modal LLMs capable of processing and generating content based on both text and visual inputs, which can be particularly useful for interpreting medical imaging data alongside textual information.

In examples, the content generation system 108 can incorporate advanced natural language processing (NLP) techniques 144, including named entity recognition and/or relationship extraction, to enhance the accuracy and/or relevance of the generated content. Additionally, the content generation system 108 can be augmented with external knowledge bases or ontologies to provide domain-specific expertise and/or ensure the generated content aligns with current medical standards and/or practices.

In examples, the content generation system 108 (and/or the service provider 102) can analyze a content item, such as a content item provided by the service provider 102 (which may have been received from the device 104 and/or otherwise obtained). For instance, a content analysis component 138 can analyze a content item (e.g., document, file, or other content) uploaded by a user using the NLP techniques 140 or other techniques to identify topics, terms discussed or related to the content item, etc. Such identified topics/terms can be used to generate content. To illustrate, a user can upload notes, a medical content item, or another content item regarding a medical condition and the content generation system 108 can analyze uploaded content item to identify topics mentioned therein. Thereafter, the content generation system 108 can generate machine-generated content for training, such as by generating terms and/or definitions of those terms for vocabulary education.

Example NLP techniques 140 and/or content analysis techniques 138 can include topic modeling, semantic analysis, and/or named entity recognition, text classification algorithms, sentiment analysis, keyword extraction to further understand the content item, machine learning techniques like clustering and/or dimensionality reduction (e.g., used to identify patterns and/or relationships within the content item). NLP techniques 140 and/or content analysis techniques 138 can include information retrieval techniques, including tf-idf (term frequency-inverse document frequency) analysis and/or latent semantic indexing, to identify key concepts and/or their relevance. In some implementations, the content analysis component 138 can incorporate computer vision techniques for analyzing visual content, such as image recognition and/or object detection, to extract information from diagrams, charts, or other visual elements within the content item.

In examples, the content generation system 108 can be configured to train a model based on data discussed herein. For example, the content generation system 108 can train a model based on analytics data received from the service provider 102 to facilitate, for example, generation of content that is most relevant to the analytics data, such as to generate content that is specific to a trending medical condition.

The content generation system 108 can be implemented as one or more computing devices, such as one or more servers, one or more desktop computers, one or more laptops computers, or any other type of device configured to process data. In some examples, the one or more computing devices are configured in a cluster, data center, cloud computing environment, or a combination thereof. In some examples, the one or more computing devices of the content generation system 108 are implemented as a remote computing resource that is located remotely. In other examples, the one or more computing devices of the content generation system 108 are implemented as local resources. The content generation system 108 can include control circuitry, data storage, one or more network interfaces, etc. The various components of the content generation system 108 can be electrically and/or communicatively coupled using certain connectivity circuitry/devices/features, which may or may not be part of the control circuitry.

The service provider 102, the device 104, data source 106, and/or content generation system 108 can be configured to communicate with each other over one or more networks. For example, the service provider 102, the device 104, data source 106, and/or content generation system 108 can send/receive data/communications/messages in a wireless and/or wired manner over one or more networks. The one or more networks can comprise one or more personal area networks (PANs), one or more local area networks (LANs), one or more wide area networks (WANs), one or more Internet area networks (IANs), one or more cellular networks, the Internet, and so on. The one or more networks can include one or more wireless and/or one or more wired networks. In some examples, the service provider 102, the device 104, data source 106, and/or content generation system 108 can implement a wireless technology, such as Bluetooth, Wi-Fi, near-field communication (NFC), etc.

In examples, the service provider 102 can provide functionality that leverages current data. For example, the service provider 102 can receive and/or process real-time/current data, such as electronic health records, laboratory results, prescription information, sensor data from medical devices, journal articles, or any other data/information discussed herein. This current data can be collected from a single healthcare facility/data source or from multiple facilities/data sources. The service provider 102 can analyze the current data to identify patterns/trends in health data, such as trends in health conditions at various scales (e.g., facility-specific, regional, or national levels). Based on these identified trends, content can be generated that reflects current healthcare patterns/trends. The content can be output to users. This approach can provide users with up-to-date information that is relevant to their specific work environment, effectively preparing them for the types of cases they are likely to encounter in their daily practice. In examples, content can be tailored to specific job roles or specialties within the healthcare field.

Further, in examples, the service provider 102 can provide adaptive content designed to guide users through an incremental educational process. This approach can facilitate progressive learning/training, such as by changing the difficulty of the training material as the user progresses. The adaptive nature of the content can allow for a more efficient and/or targeted educational experience, improving knowledge retention and/or practical application of the learned material. In some cases, content can be generated that is tailored to specific hospital, clinics, medical specialties, organization, entity, etc.

Furthermore, in examples, the service provider 102 can facilitate prioritized learning/training, wherein machine-generated content is generated for frequent topics, medical conditions/diagnosis, etc. that are experienced/observed in a context. For instance, the service provider 102 can identify a medical condition that is most frequently diagnosed (or a top number of diagnosed medical conditions) in a hospital based on analytics data. The service provider 102 can cause machine-generated content to be generated for that medical condition(s). In one illustration where the machine-generated content is training content, a user can learn about medical conditions that are most frequently observed at a specific hospital, geographical area, job position, department, etc. As such, the user can receive tailored training for the user's specific context to quickly train the user on scenarios that will be most likely experienced.

Moreover, in examples, the service provider 102 can provide different machine-generated content in response to each data generation request. For example, the service provider 102 can cause first machine-generated content to be generated for a first data generation request related to a topic, and thereafter cause second machine-generated content to be generated for a second data generation request related to the same topic. Here, the first and second content can be different, which can be due to differences in the data generations requests and/or due to the somewhat random nature of the content generation system 108 (e.g., using a stochasticity language model, primarily probabilistic sampling, nondeterminism, etc.). This can increase the breadth of machine-generated content that is presented to a user, which can be helpful in training or other uses (e.g., provide various opportunities to learn about the same topic).

FIG. 2 illustrates additional example details of the service provider 102 of FIG. 1 according to one or more examples. The service provider 102 can be implemented as one or more computing devices, such as one or more servers, one or more desktop computers, one or more laptops computers, or any other type of device configured to process data. In some examples, the one or more computing devices are configured in a cluster, data center, cloud computing environment, or a combination thereof. In some examples, the one or more computing devices of the service provider 102 are implemented as a remote computing resource that is located remotely to another device. In other examples, the one or more computing devices of the service provider 102 are implemented as local resources that are located locally. As illustrated, the service provider 102 can include control circuitry 202, data storage 204, one or more network interfaces 206, a conversation component 208, analytics component 210, anonymization component 110, content verification component 112, data request component 114, and visualization component 116. The various components of the service provider 102 can be electrically and/or communicatively coupled using certain connectivity circuitry/devices/features, which may or may not be part of the control circuitry 202.

The control circuitry 202 can include one or more processors, such as one or more central processing units (CPUs), one or more microprocessors, one or more graphics processing units (GPUs), one or more digital signal processors (DSPs), and/or other processing circuitry. Alternatively, or additionally, the control circuitry 202 can include one or more application specific integrated circuits (ASIC), one or more field-programmable gate arrays (FPGAs), one or more program-specific standard products (ASSPs), one or more complex programmable logic devices (CPLDs), and/or the like. The control circuitry 202 can be configured to execute one or more instructions stored in the data storage 204 to thereby perform one or more operations to implement various functionality discussed herein. The control circuitry 202 can operate in cooperation with any of the components of the service provider 102 to facilitate such functionality.

The data storage 204 can include any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data. Computer-readable media that may be implemented in accordance with examples of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media. In examples, the data storage 204 may store one or more instructions that are executable by the control circuitry 202 to facilitate various functionality discussed herein.

In some examples, the conversation component 208, analytics component 210, anonymization component 110, content verification component 112, data request component 114, and visualization component 116 may be implemented as one or more instructions stored in the data storage 204, which are configured to be executed by the control circuitry 202 to perform various operations discussed herein. Further, in some examples, the conversation component 208, analytics component 210, anonymization component 110, content verification component 112, data request component 114, and visualization component 116 can be implemented as one or more instructions stored in the data storage 204 that is implemented as one or more application specific integrated circuits (ASIC), one or more field-programmable gate arrays (FPGAs), one or more program-specific standard products (ASSPs), one or more complex programmable logic devices (CPLDs), and/or the like. For ease of discussion, the conversation component 208, analytics component 210, anonymization component 110, content verification component 112, data request component 114, and visualization component 116 may be implemented as one or more instructions stored in the data storage 204 are illustrated as separate components. However, such components may be implemented as any number of components to implement the functionality discussed herein (e.g., combined or separated into additional components).

The one or more network interfaces 206 can be configured to communicate with one or more devices over a communication network. For example, the one or more network interfaces 206 may send/receive data in a wireless or wired manner over a network. A communication network can include a local area network (LAN), a wide area network (WAN) (e.g., the Internet), a personal area network (PAN), a body area network (BAN), etc. In some examples, the one or more network interfaces 206 may implement a wireless technology such as Bluetooth, Wi-Fi, near field communication (NFC), or the like. In some examples, the one or more network interfaces 206 may include a transceiver (e.g., transceiver circuitry embodied in one or more devices) configured to transmit/receive signals wirelessly.

The conversation component 208 can be configured to facilitate a conversation between a user and a chatbot, such as an AI chatbot, virtual assistant, conversational AI, voice bot, or other chatbot. For example, the conversation component 208 can implement a rule-based chatbot that follows predefined scripts and/or decision trees to respond to user inputs. Alternatively, or additionally, the conversation component 208 can utilize a retrieval-based chatbot that selects appropriate responses from a database of pre-written replies. More advanced implementations can include generative AI chatbots powered by LLMs, capable of producing dynamic, context-aware responses. In examples, the conversation component 208 is implemented in cooperation with (e.g., with the assistance of) the content generation system 108. The conversation component 208 can also support hybrid chatbots that combine multiple approaches, such as using both retrieval and generative methods. In some cases, the conversation component 208 can facilitate interactions with embodied conversational agents or virtual assistants that use avatars and/or NLP to create immersive experiences. In some instances, the conversation component 208 can incorporate voice-enabled chatbots that can engage in spoken conversations through speech recognition and/or text-to-speech technologies. Additionally, or alternatively, the conversation component 208 can support multi-modal chatbots that can process and/or respond to various input types, including text, voice, images, and/or gestures, providing a more versatile interaction experience. In some examples, the conversation component 208 can provide output that simulates a human voice.

In one illustration, the conversation component 208 (which can operate in cooperation with any of the other components of the service provider 102, content generation system 108, and/or device 104) can facilitate a conversation between a user and a chatbot to educate a user on a certain topic. In one case, the chatbot can provide an example patient scenario (e.g., including details about the patient's condition) and the user can respond with a recommendation/approach for handling the patient scenario. Based on the user's response, the chatbot can provide an answer regarding the user's response (e.g., an indication if the user's response was an appropriate recommendation). The chatbot can also provide information about an optimal/best recommendation/approach for handling the patient scenario. The conversation between the user and the chatbot can include any number of responses (e.g., in a back-and-forth manner).

In another illustration, the conversation component 208 (which can operate in cooperation with any of the other components of the service provider 102, content generation system 108, and/or device 104) can facilitate a verbal conversation with a user. In one case, the conversation component 208 can provide output that simulates the voice of a family member of a patient to train a user on responding to questions that may be asked by family members while the patient is in the hospital. Here, a nurse or another user that is interacting with the device 104 to receive training can have a conversation with the family member to explain what is occurring to the patient (e.g., in a simulated manner). In some instances, the conversation can occur entirely over voice/audio input/output. However, other input/output methods can be implemented.

The analytics component 210 can be configured to analyze the patient data 120, sensor data 122, medical content items 212, and/or other data to generate/create analytics data 214 related to trends/metrics/analytics in medical conditions, protocols/guidelines/standards for treating medical conditions, etc. For example, the analytics data 214 can include/indicate trends in medical conditions for a particular geographic area, a top number of medical conditions that are treated, a medical condition that is most frequently observed by a certain type of medical professional, a medical condition that is most frequently diagnosed in a certain geographic area, etc.

Example analytics data can include/indicate:
Trends in medical conditions. In some cases, trends can be based on identifying emerging health issues, tracking the spread of diseases, or monitoring changes in prevalence of certain conditions over time.
Metrics related to healthcare delivery and/or outcomes, such as treatment efficacy rates, patient recovery times, or healthcare resource utilization.
Geographical trends, such as patterns in medical conditions specific to particular regions, cities, neighborhoods, etc.
Most common conditions, such as the top number of medical conditions that are treated in a given healthcare system, region, etc.
Profession-specific insights, such as which medical conditions are most frequently observed by certain types of medical professionals.
Location-specific diagnoses, such as which medical conditions are most frequently diagnosed in specific geographic areas.
Temporal trends, such as how medical conditions change over time.
Demographic patterns, such as how medical conditions vary across different age groups, genders, or other demographic categories.
Treatment efficacy, such as which treatments are most effective for specific conditions.
Resource utilization, such as patterns in how healthcare resources are used.
Analytics on various aspects of healthcare, such as a wide range of analyses, from patient satisfaction scores to cost-effectiveness of different treatment protocols.

In examples, the analytics data 214 can be used by the conversation component 208, anonymization component 110, content verification component 112, data request component 114, and/or visualization component 116. In one illustration, the data request component 114 can use the analytics data 214 to generate a data generation request that is specific to a particular trend, such as by requesting that content be generated that is related to a medical diagnosis that is frequently occurring, a medical diagnosis that is most frequently occurring in a geographical area, etc. In another illustration, the content verification component 112 can use the analytics data 214 to verify that machine-generated content aligns with current trends in medical conditions, protocols/guidelines/standards for treating medical conditions, etc. In yet another illustration, the content verification component 112 and/or the visualization component 116 can update/reorganize/convert or otherwise manipulate machine-generated content to reflect the analytics data 214, such as by ranking content in the machine-generated content in a particular order. In a further illustration, the content verification component 112 and/or the visualization component 116 can analyze/process the analytics data 214 to identify medical conditions that are most common in a particular geographical area, most seen by a particular medical professional (e.g., medical conditions that nurses are treating the most in patients), etc. The output data 136 can relate to the analytics data 214. To illustrate, a nurse that is associated with a particular department within a hospital may desire to receiving training on medical conditions that are most commonly treated within that department.

In examples, the analytics component 210 is configured to operate in cooperation with the visualization component 116 to generate a map, chart, rankings, and/or another visualization/information regarding the analytics data 214. In one illustration, the visualization component 116 can process the analytics data 214 to generate a heat map or another map for a geographical region to indicate concentrations of a medical condition across the geographical region. In another illustration, a user can provide input for a specific geographical region or hospital that the user may be visiting and the visualization component 116 can process the analytics data 214 to generate a ranking of top medical conditions in that geographical region or hospital.

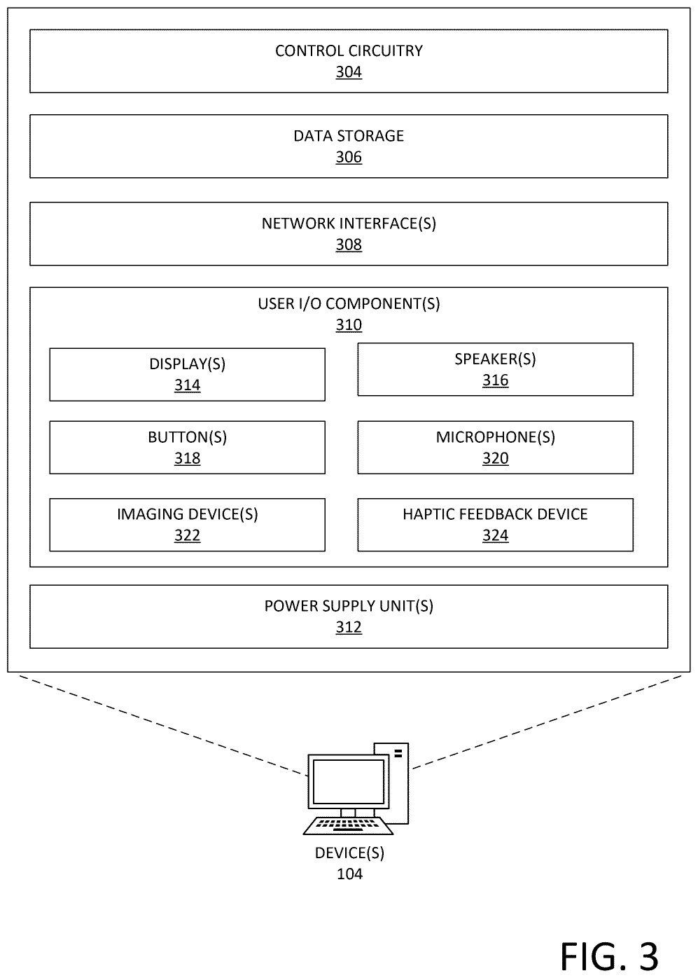

FIG. 3 illustrates details of the device 104 of FIG. 1 according to one or more examples. The device 104 can be implemented as one or more computing devices, such as one or more desktop computers, one or more laptops computers, one or more servers, one or more smartphones, one or more electronic reader devices, one or more mobile handsets, one or more personal digital assistants, one or more portable navigation devices, one or more portable gaming devices, one or more tablet computers, one or more wearable devices (e.g., a watch), one or more portable media players, one or more televisions, one or more set-top boxes, one or more computer systems in a vehicle, one or more appliances, one or more cameras, one or more security systems, one or more home-based computer systems, one or more projectors, one or more virtual reality devices/headsets, one or more augmented reality devices/headsets, one or more spatial computers/headsets, and so on. As illustrated, the device 104 can include control circuitry 304, data storage 306, one or more network interfaces 308, one or more user input/output components 310, and one or more power supply unit(s) 312. The various components of the device 104 can be electrically and/or communicatively coupled using certain connectivity circuitry/devices/features, which may or may not be part of the control circuitry 304.

The control circuitry 304 can include one or more processors, such as one or more central processing units (CPUs), one or more microprocessors, one or more graphics processing units (GPUs), one or more digital signal processors (DSPs), and/or other processing circuitry. Alternatively, or additionally, the control circuitry 304 can include one or more application specific integrated circuits (ASIC), one or more field-programmable gate arrays (FPGAs), one or more program-specific standard products (ASSPs), one or more complex programmable logic devices (CPLDs), and/or the like. The control circuitry 304 can be configured to execute one or more instructions stored in the data storage 306 to thereby perform one or more operations to implement various functionality discussed herein. The control circuitry 304 can operate in cooperation with any of the components of the device 104 to facilitate such functionality.

The data storage 306 can include any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data. Computer-readable media that may be implemented in accordance with examples of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media. The data storage 306 may store one or more instructions that are executable by the control circuitry 304 to facilitate various functionality discussed herein.

The one or more network interfaces 308 can be configured to communicate with one or more devices over a communication network. For example, the one or more network interfaces 308 may send/receive data in a wireless or wired manner over a network. A communication network can include a local area network (LAN), a wide area network (WAN) (e.g., the Internet), a personal area network (PAN), a body area network (BAN), etc. In some examples, the one or more network interfaces 308 may implement a wireless technology such as Bluetooth, Wi-Fi, near field communication (NFC), or the like. In some examples, the one or more network interfaces 308 can include a transceiver (e.g., transceiver circuitry embodied in one or more devices) configured to transmit/receive signals wirelessly.

The one or more user I/O components 310 can include one or more electronic displays 314 configured to display data, one or more speakers 316 configured to output an audio signal, one or more buttons 318 configured to receive input, one or more microphones 320 configured to detect sound and convert the sound into an electrical signal, one or more imaging devices 322, one or more haptic feedback devices 324 configured to produce various types of haptic sensations, including vibrations, forces, textures, or motions (to provide tactile information to the user). The one or more displays 314 can include one or more liquid-crystal (LCD) displays, light-emitting diode (LED) displays, organic LED displays, plasma displays, electronic paper displays, and/or any other type of technology. In some examples, the one or more displays 314 include one or more touchscreens configured to receive input and/or display data. The one or more buttons 318 can include one or more mechanical push-buttons configured to be depressed or pushed, one or more touch buttons configured to receive touch input, or any other type of button. The one or more imaging devices 322 can include one or more cameras configured to capture an image(s) of an environment, one or more depth/range sensors configured to generate depth information for an environment, and so on. Although the one or more user I/O components 310 are illustrated as separate components, any of the components can be implemented together.

The one or more user I/O components 310 can be configured to receive input from a user and/or provide output to the user. In one example, the one or more user I/O components 310 provide a user interface via the one or more displays 314, and a user can provide input via the user interface. In some examples, a user can provide speech input via the microphone(s) 320 and the device 104 and/or another device can perform operations requested by the speech input. In some examples, a user can provide input via the one or more displays 314/the one or more buttons 318, and the device 104 and/or another device can perform an operation based on the input. In some examples, the one or more user I/O components 310 or other components can operate to receive gesture input (e.g., the imaging device(s) 322 can capture an image(s) of a user making a gesture(s) and the gesture(s) can be processed using gesture recognition to identify an operation being requested). In examples, the user I/O components 310 can be configured to provide simulated output, such as to simulate how to treat a medical condition of a patient.

In one illustration, the one or more user I/O components 310 can be configured to provide a simulate environment. For example, the service provider 102 can generate (or cause to be generated) content that simulates a patient scenario, which can include a simulated patient room/environment or hospital. In some cases, the service provider 102 generates such content based on uploaded videos/images of a hospital environment. The service provider 102 can provide the content to the device 104, wherein the content can include simulated patient scenario content (e.g., diagnosis details) and/or simulated environment content. The one or more user I/O components 310 can output the simulated environment content to create a simulate hospital environment. Further, the one or more user I/O components 310 can output the simulated patient content. In some cases, the simulated patient content includes content that simulates the appearance of a patient, such as an avatar/virtual avatar. Thereby, a user of the device 104 can interact with the simulated environment and patient scenario, such as to train the user on handling such situation.

The power supply unit(s) 312 can be configured to manage power for the device 104, such as power provided to and/or received from one or more components of the device 104. In some examples, the power supply unit(s) 312 includes one or more batteries, such as a lithium-based battery, a lead-acid battery, an alkaline battery, and/or another type of battery. That is, the power supply unit(s) 312 can comprise one or more devices and/or circuitry configured to provide a source of power and/or provide power management functionality. Further, in some examples, the power supply unit(s) 312 includes a mains power connector that is configured to couple to an alternating current (AC) or direct current (DC) mains power source.

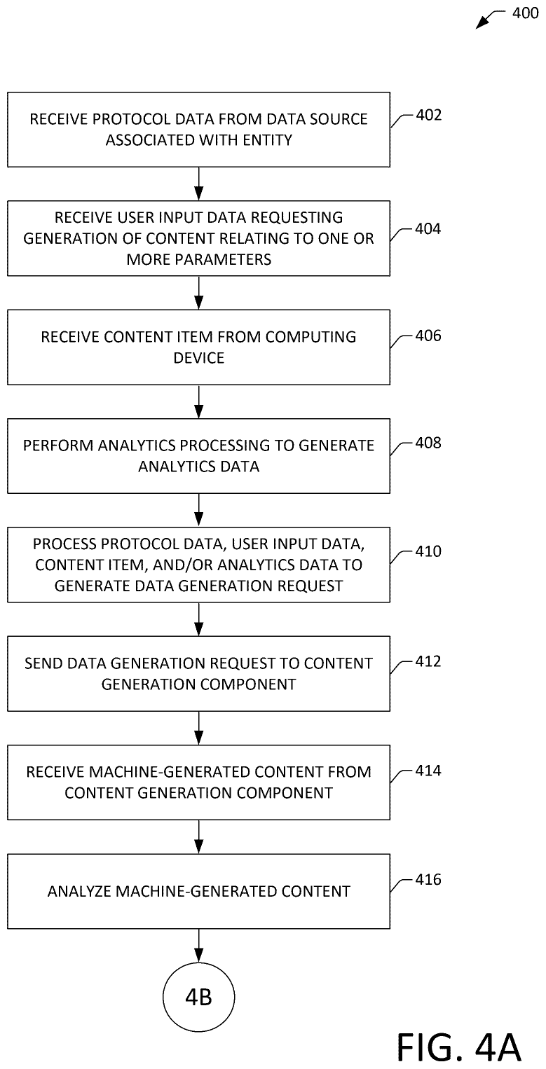

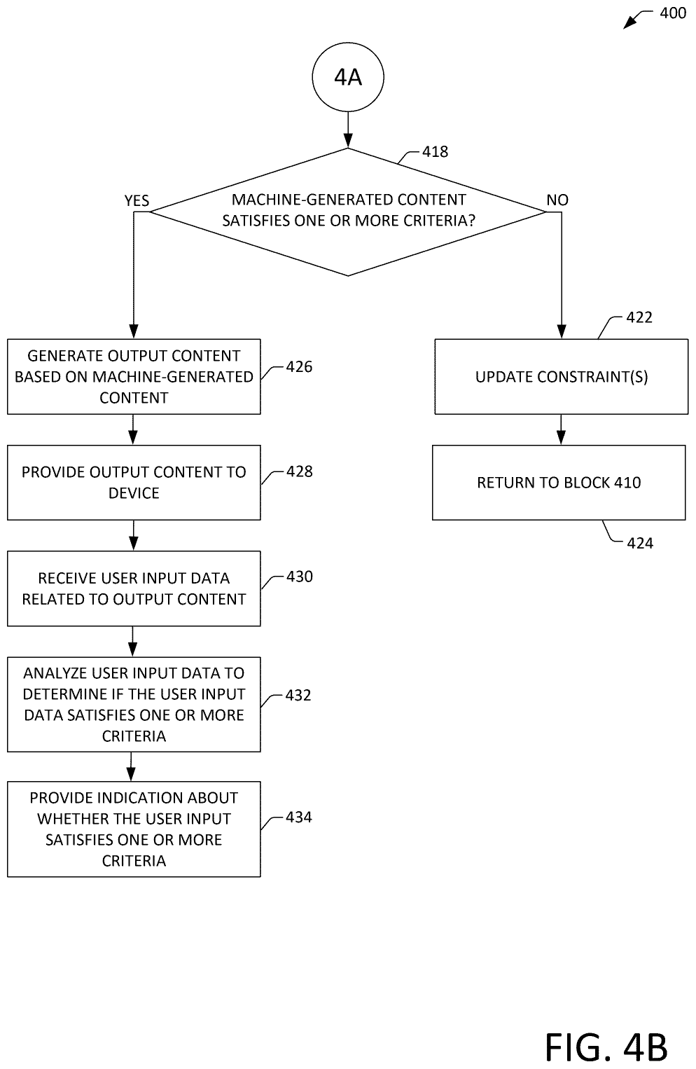

FIGS. 4A and 4B illustrate an example process 400 in accordance with one or more examples. For ease of illustration, the process 400 can be performed in the example architecture 100 of FIG. 1. For example, one or more of the individual operations of the process 400 can be performed by the service provider 102 (e.g., the control circuitry 202 of the service provider 102), the content generation system 108, the device 104 (e.g., the control circuitry 304 of the device 104), the data source 106, etc. However, the process 400 can be performed in other architectures and/or by other devices. Moreover, the architecture 100 can be used to perform other processes.

The process 400 (as well as each process described herein) is illustrated as a logical flow graph, each graph of which represents a sequence of operations that can be implemented in hardware, software, or a combination thereof. In the context of software, the operations represent executable instructions stored on one or more computer-readable media that, when executed by control circuitry (e.g., one or more processors or other components), perform the recited operations. Generally, executable instructions include routines, programs, objects, components, data structures, and the like that cause particular functions to be performed or implement particular data types. The order in which the operations are described is not intended to be construed as a limitation, and any number of the described operations can be combined in any order and/or in parallel to implement a process. Further, any number of the described operations can be omitted.

In FIG. 4A, at block 402, the process 400 includes receiving protocol data from a data source associated with an entity. For example, the service provider 102 can receive protocol data from the data source 106. The protocol data can include/indicate a procedure(s), guideline(s), policy(ies), standard(s), etc., which can be defined by the entity.

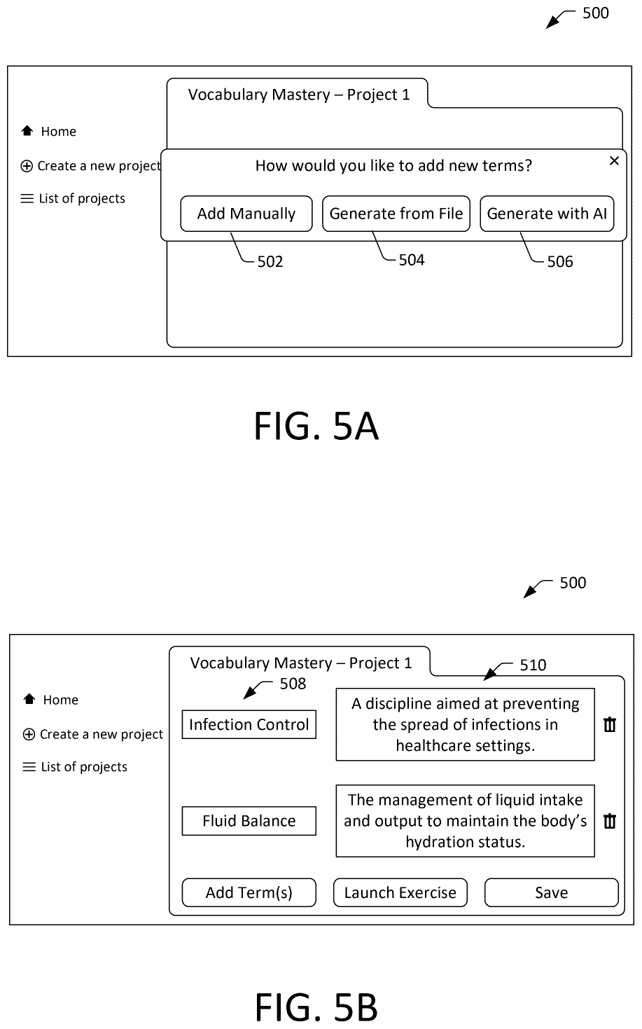

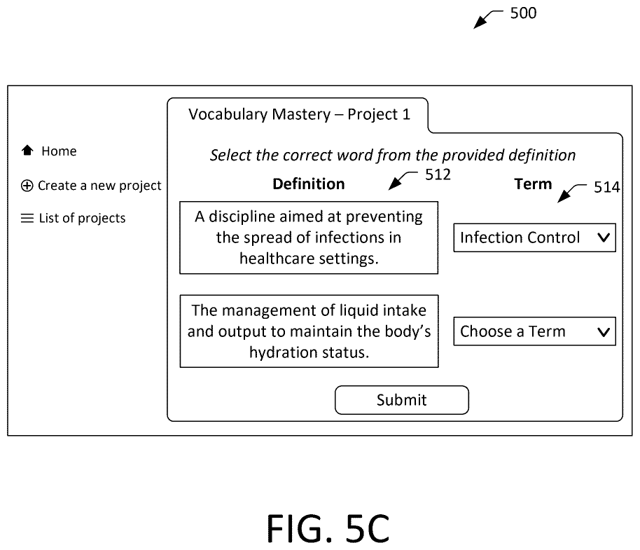

At block 404, the process 400 includes receiving user input data requesting generation of content relating to one or more parameters. For example, the service provider 102 can receive user input data from the device 104 requesting content based on one or more parameters. In some cases, a parameter includes a specific keyword, phrase, or concept/topic, which can allow for more focused content generation. Further, in some cases a parameter can include/indicate a medical condition or disease, treatment method or procedure, class of medications or therapeutic interventions, area of medical specialization, type of medical equipment or technology, healthcare setting (e.g., emergency room, intensive care unit, outpatient clinic, etc.), patient demographic group (e.g., pediatric, geriatric, pregnant women, etc.), timeframe or historical period in medical practice, geographical region or healthcare system, specific medical test or diagnostic procedure, healthcare policy or regulation, public health initiative or program, medical research topic or clinical trial, ethical issue in healthcare, medical specialty or subspecialty, type of healthcare provider role (e.g., nurse, physician assistant, specialist, etc.), medical emergency or crisis situation, preventive care measure or wellness program, healthcare technology or innovation, patient care protocol, etc. In some instances, the user input data is part of a conversation between a user and a chatbot, such as a question/answer, response, etc. In some instances, the user input data specifies a certain type of training, such as vocabulary training, medication training, clinical training, etc. In some examples, the user input data relates to one of the user interfaces shown in FIGS. 5-8.

At block 404, the process 400 includes receiving a content item from a computing device. For example, the service provider 102 can receive a content item from the device 104 (e.g., an upload of the content item, identifying information to retrieve the content item, etc.). In some cases, the user input data received at block 404 includes a request to analyze the content item, such as to identify terms within the content item or concepts expressed therein to thereby generate content related to such terms/concepts. In one illustration, a user can upload a content item to be analyzed for generating content, such as to generate vocabulary terms related to notes uploaded by the user. In another illustration, a user can upload a medical content item to cause analytics data to be generated for the medical content item.

At block 408, the process 400 includes performing analytics processing. For example, the service provider 102 can receive patient data, sensor data, medical content item, and/or other data from the device 104, data source 106, and/or another device/component. The service provider 102 can process the data/item to generate analytics data related to trends/metrics/analytics in the data, protocols for treating medical conditions, etc.

At block 410, the process 400 includes processing protocol data, user input data, content item, and/or analytics data to generate a data generation request. For example, the service provider 102 can process/analyze protocol data, user input data, content item, and/or analytics data to generate a data generation request that includes a request (e.g., for the content generation system 108) to generate content, such as content related to the user input data, content item, protocol data, analytics data, etc. In examples, the data generation request includes one or more constraints that are based on protocol data, user input data, content item, and/or analytics data.

At block 412, the process 400 includes sending a data generation request to a content generation component. For example, the service provider 102 can send a data generation request to the content generation system 108, wherein the data generation request can include one or more constraints for generating content. In examples, the data generation request includes/identifies a content item and/or includes a request to analyze the content item and/or generated content based on the analysis.

At block 414, the process 400 includes receiving machine-generated content from a content generation component. For example, the service provider 102 can receive machine-generated content from the content generation system 108. In some illustrations, machine-generated content can include clinical data related to treating a medical condition, simulated laboratory results for a sample patient scenario, medication data related to treating a medical condition, etc.

At block 416, the process 400 includes analyzing machine-generated content. For example, the service provider 102 can process/analyze machine-generated content in relation to protocol data, user input data, a constraint (e.g., from a data generation request), data generation request, and/or analytics data to evaluate if the machine-generated content satisfies one or more criteria. The one or more criteria can be established based on protocol data, user input data, a constraint, data generation request, and/or analytics data to evaluate the machine-generated content to ensure that the machine-generated content aligns with the one or more criteria.

In FIG. 4B, at block 418, the process 400 includes determining whether the machine-generated content satisfies one or more criteria. For example, based on the analysis at block 416, the service provider 102 can determine whether the machine-generated content satisfies the one or more criteria (e.g., evaluate if the content generation system 108 provided content that aligns with the data generation request, protocol data, user input data, analytics data, constraint, etc.).

In response to determining that the machine-generated content does not satisfy the one or more criteria, the process 400 can proceed to block 422 (i.e., the “NO” route). At block 422, the process 400 includes updating one or more constraints for including in a data generation request. At block 424, the process 400 can then return to block 410 in FIG. 4A to regenerate another data generation request with the updated constraint(s). By doing so, the process 400 can regenerate another data request that includes updated constraints in an attempt to receive additional machine-generated content that satisfies the one or more criteria (e.g., better aligns with aligns with protocol data, user input data, analytics data, constraint, etc.). In examples, an updated constraint includes an additional constraint, a narrower/more limiting constraint than a previous constraint, etc.

In response to determining that the machine-generated content satisfies the one or more criteria, the process 400 can proceed to block 426 (i.e., the “YES” route). In some cases, the one or more criteria is established such that the one or more criteria are satisfied if the machine-generated content is close enough (e.g., above a threshold, more than a threshold number of criteria are satisfied, etc.). In examples, the service provider 12 can update the machine-generated content such that the machine-generated content satisfies the one or more criteria.

At block 426, the process 400 can include generating output content based on machine-generated content. For example, the service provider 102 can generate output content, such as audio output, displayable content, haptic output, etc., based on machine-generated content. In examples, displayable content includes graphical elements for a user interface; however, other displayable items can be generated.

At block 428, the process 400 includes providing output content to a device. For example, the service provider 102 can send the output data to the device 104, such as to display the output data via the device 104. In some examples, generating data for display, sending the data to another device for display on the other device, and/or displaying the data may be referred to as “causing display of the data.”

At block 430, the process 400 includes receiving user input data related to output content. For example, the service provider 102 can receive user input data based on a user viewing/receiving/hearing the output data. To illustrate, the user can provide a response to a question, ask a question, or otherwise provide input to interact with a chatbot or other system.

At block 432, the process 400 includes analyzing/processing user input data related to output content to determine if the user input data satisfies one or more criteria. For example, the service provider 102 can analyze the user input data (e.g., that is based on the user viewing the output data) to determine if the user input data satisfies one or more criteria, such as the user input data providing the correct response to a question/scenario. In examples, the one or more criteria are based on the machine-generated content (e.g., an answer that is generated by the content generation system 108, but not provided to the user). In examples, the service provider 102 processes the user input data without communicating with the content generation system 108 (e.g., determines the correct or incorrect answer based on its own data or data that was previously provided to the service provider 102 from the content generation system 108). In other examples, the service provider 102 communicates with the content generation system 108 where the user input data is received to evaluate the user input data (e.g., the content generation system 108 performs an analysis of the user input data).

At block 434, the process 400 includes providing an indication about whether the user input data satisfies the one or more criteria. For example, the service provider 102 can provide an indication (e.g., within output data) to the device 104 that the user input data did or did not satisfy the one or more criteria (e.g., an indication that the user provided the wrong or right answer). In one illustration, an indication is provided as a response from a chatbot.

In examples, the process 400 can be performed any number of times to provide different content. To illustrate, during a first interaction, a user can interface with the service provider 102 to receive training regarding a certain topic, such as by providing answers to questions that are asked by a chatbot regarding the topic. During a second interaction, the user can interface with the service provider 102 again to receiving training regarding the topic. In this instance, the service provider 102 can provide different information to the user (although generally similar), even if the request from the user is the same. For instance, the service provider 102 can cause slightly different content to be generated for the user, wherein such content can be different due to the dynamic/generative nature of the content generation system 108, updated content that may be referenced (e.g., more current information at the time of the second interaction), and/or a new data generation request provided by the service provider 102. In some cases, the service provider 102 can cause more advanced content to be generated for a user based on having previously determined that the user mastered certain topics (e.g., provided a certain number of correct answers). As such, the user can receive machine-generated content that dynamically changes over time, which can enhance the user's understanding.

FIGS. 5-8 illustrate example interfaces that can be presented according to one or more examples. The interfaces (also referred to as “user interfaces”) can be provided via a web browser, an application (e.g., a mobile application, desktop application etc.), and so on. These interfaces are provided for illustrative purposes and other types of interfaces can be provided to facilitate one or more of the operations discussed herein. Although the interfaces include certain elements and/or the elements are arranged in certain manners, any of such elements can be changed/eliminated and/or the elements can be arranged differently. Further, although examples elements are shown with respect to a particular interface, any of the elements can be combined in different arrangements. In examples, in any of the contexts of FIGS. 5-8 (or other contexts discussed herein), a user can create a project for training for education purposes or other purposes, which the user can return to after creating to complete, review, etc. The examples of FIGS. 5-8 illustrate some of many example projects and/or interfaces that can be created/displayed.

FIGS. 5A, 5B, and 5C illustrate an example interface 500 for facilitating vocabulary mastery/training using one or more of the techniques discussed herein. For examples, in FIG. 5A, the interface 500 can enable the user to select from several options for generating a vocabulary quiz. In a first option, the user can select a button/icon/interface element 502 to manually enter terms and definitions. In a second option, the user can select a button/icon/interface element 504 to generate terms and definitions by having the service provider 102 analyze a content item (e.g., file) that is uploaded by the user. Here, the user can upload a content item and the service provider 102 can analyze the content item to identify terms therein and/or concepts expressed therein. The service provider 102 can then cause machine-generate terms and/or definitions to be generated. In a third option, the user can select a button/icon/interface element 506 to generate terms using AI without adding them manually or uploading a content item. Here, the user can specific a topic, medical condition, or any parameter and the service provider 102 can cause machine-generate terms and/or definitions to be generated that are related to that parameter.

FIG. 5B illustrates the example interface 500 with vocabulary terms 508 and definitions 510, wherein the vocabulary terms 508 and/or definitions 510 can be manually and/or machine-generated. The user can launch the exercise (e.g., quiz/test), causing the interface 500 of FIG. 5C to be presented. The interface 500 of FIG. 5C includes definitions 512 and drop-down menus 514 for the user to choose a term that matches the definition. Once submitted, the interface 500 can also provide an indication of whether the user matched the definitions to the correct terms. In examples, rationale/reasoning for the correct/incorrect answer can be provided.

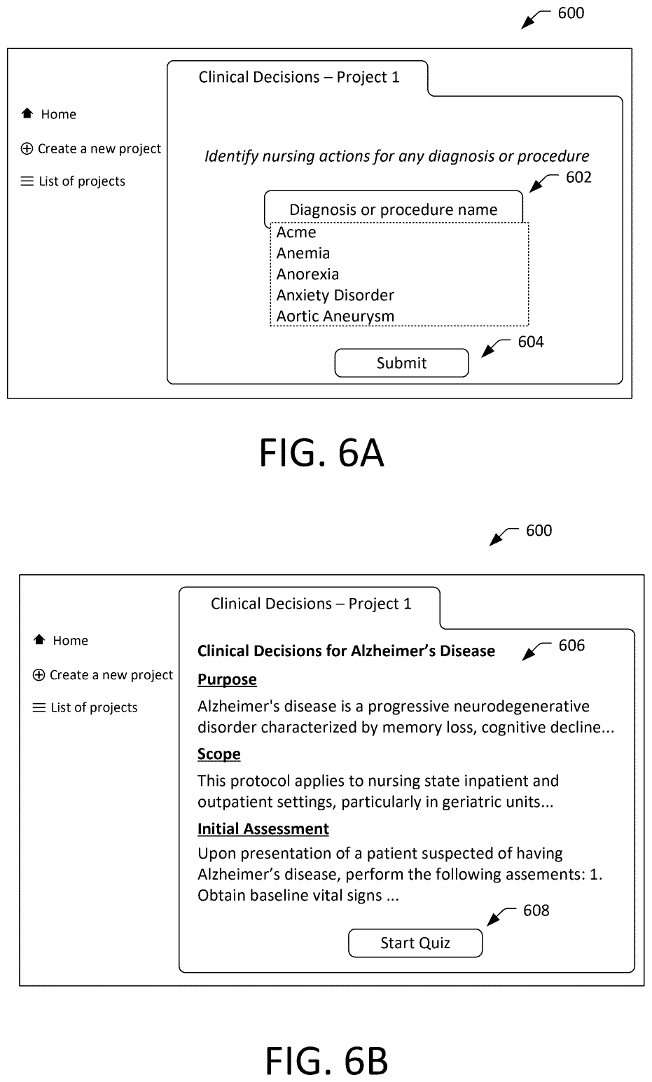

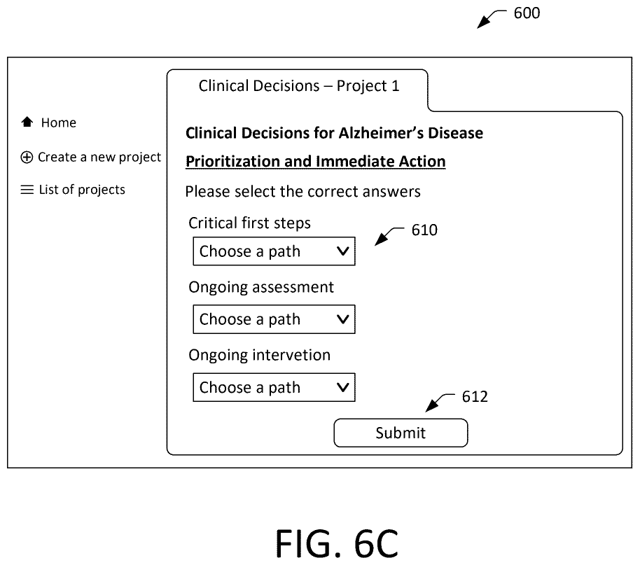

FIGS. 6A, 6B, and 6C illustrate an example interface 600 for facilitating clinical training using one or more of the techniques discussed herein. In FIG. 6A, the interface 600 enables a user to specify a diagnosis or procedure to study in an input field 602. Upon receiving input from the user and a selection of a button 604, the service provider 102 can cause machine-generated content to be generated related to the diagnosis or procedure input. The interface 600 of FIG. 6B illustrates the machine-generated content 606 that has been generated in this example, which indicates various details about the diagnosis or procedure. The user can start the quiz upon reviewing the information and selecting a button 608. In response, the interface 600 of FIG. 6C can be presented. As shown in FIG. 6C, the user can provide answers to the questions, using drop-down menus 610 in this example. The drop-down menus 610 can include correct and incorrect options, which can be machine-generated. Upon completing the quiz (e.g., selecting a submit button 612), the user can view the results of the quiz (e.g., an evaluation of whether the user answered each of the questions correctly or incorrectly).

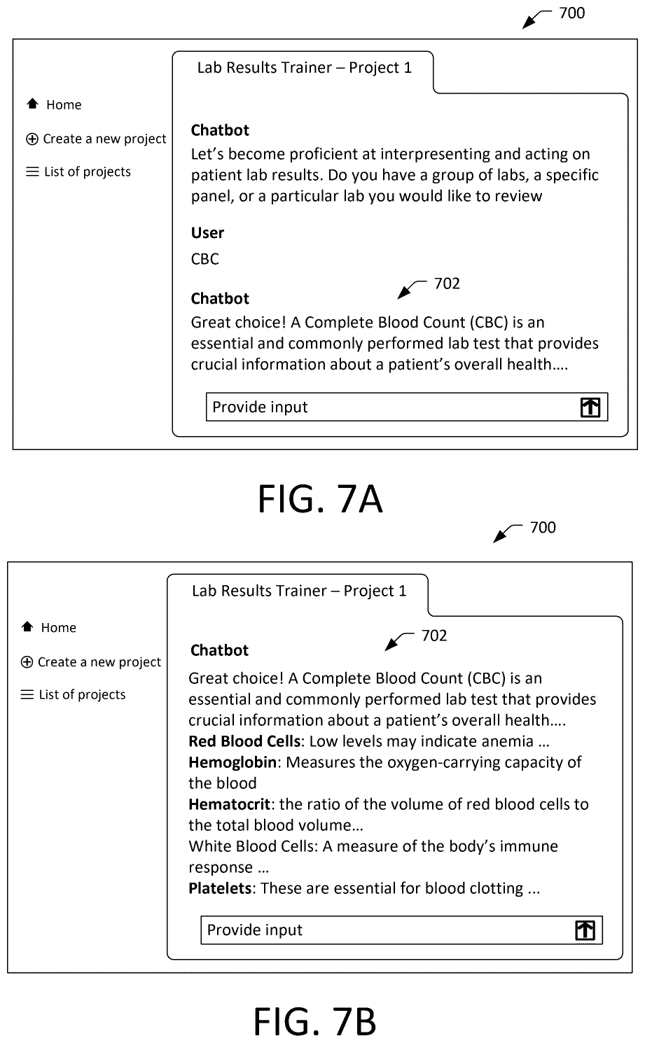

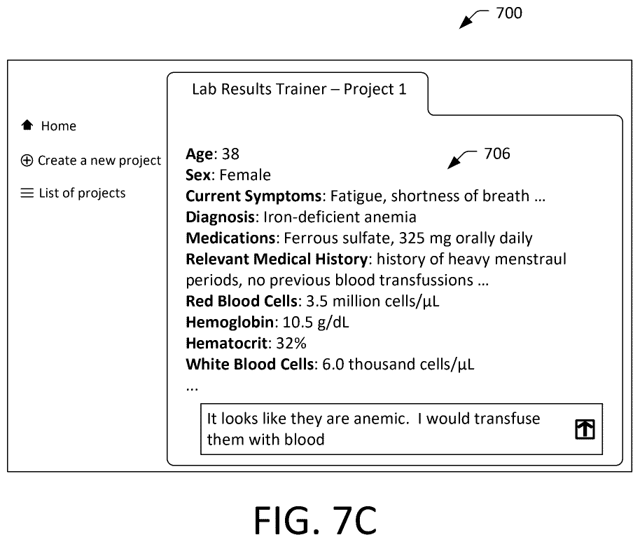

FIGS. 7A, 7B, and 7C illustrate an example interface 700 for training a user on lab tests using one or more of the techniques discussed herein. In the examples of FIGS. 7A-7C (and/or any other interface discussed herein), the user can converse with a chatbot through the interface 700. In FIGS. 7A and 7B, the interface 700 enables a user to specify a type of lab test the user would like to learn about and review information about the type of lab test. Here, the interface 700 displays information 702 regarding the type of lab test, so that the user can understand more about the lab test. The information can be machine-generated.

FIG. 7C illustrates the example interface 700 for training a user in relation to a sample patient scenario. Here, the interface 700 presents information 706 regarding lab results for a sample patient scenario. The information can be machine-generated. The user can respond with information regarding a diagnosis of the patient, a recommendation for taking action in relation to the patient, etc. The interface 700 can process the user's response and provide feedback indicating whether the user's response is valid in view of the patient scenario (e.g., an appropriate action/input).

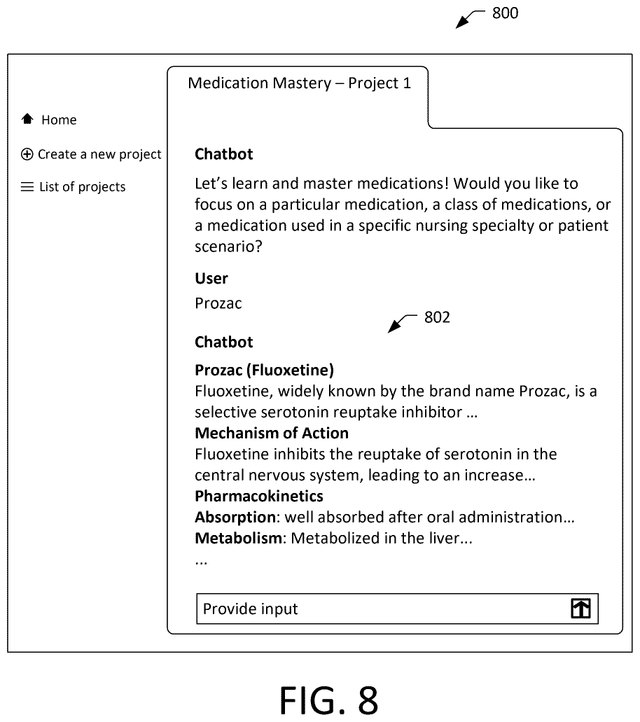

FIG. 8 illustrates an example interface 800 for training a user on a medication using one or more of the techniques discussed herein. For example, the interface 800 can enable a back-and-forth conversation with a chatbot, as similarly discussed elsewhere herein, wherein the chatbot provides a questions, response/answer, or other content (which can be machine generated in real-time) and/or the user provides a response/input regarding medication administration, usage, etc. Here, the interface 800 provides information 802 regarding a medication.

### Additional Features and Examples

The above description of examples of the disclosure is not intended to be exhaustive or to limit the disclosure to the precise form disclosed above. While specific examples, and examples, are described above for illustrative purposes, various equivalent modifications are possible within the scope of the disclosure, as those skilled in the relevant art will recognize. For example, while processes or blocks are presented in a given order, alternative examples may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel or may be performed at different times.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

In examples, certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited. In some contexts, description of an operation or event as occurring or being performed “based on,” or “based at least in part on,” a stated event or condition can be interpreted as being triggered by or performed in response to the stated event or condition.

With respect to the various methods and processes disclosed herein, although certain orders of operations or steps are illustrated and/or described, it should be understood that the various steps and operations shown and described may be performed in any suitable or desirable temporal order. Furthermore, any of the illustrated and/or described operations or steps may be omitted from any given method or process, and the illustrated/described methods and processes may include additional operations or steps not explicitly illustrated or described.

In the above description of examples, various features are sometimes grouped together in a single example, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the disclosure herein disclosed and claimed below should not be limited by the particular examples described above but should be determined only by a fair reading of the claims that follow.

Unless the context clearly requires otherwise, throughout the description and the claims, the terms “comprise,” “comprising,” “have,” “having,” “include,” “including,” and the like are to be construed in an open and inclusive sense, as opposed to a closed, exclusive, or exhaustive sense; that is to say, in the sense of “including, but not limited to.”

The word “coupled”, as generally used herein, can refer to two or more elements that may be physically, mechanically, and/or electrically connected or otherwise associated, whether directly or indirectly (e.g., via one or more intermediate elements, components, and/or devices.

Additionally, the words “herein,” “above,” “below,” and words of similar import, when used in this application, shall refer to this application as a whole, including any disclosure incorporated by reference, and not to any particular portions of the present disclosure. Where the context permits, words in present disclosure using the singular or plural number may also include the plural or singular number, respectively.

As may be used herein, the terms “substantially” and “approximately” provide an industry-accepted tolerance for its corresponding term and/or relativity between items. For some industries, an industry-accepted tolerance is less than one percent, while for other industries, the industry-accepted tolerance may be 10 percent or more. Other examples of industry-accepted tolerances range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than approximately +/−1%). Some relativity between items may range from a difference of less than a percentage level to a few percent. Other relativity between items may range from a difference of a few percent to magnitude of differences.

One or more examples have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

The one or more examples are used herein to illustrate one or more aspects, one or more features, one or more concepts, and/or one or more examples. A physical example of an apparatus, an article of manufacture, a machine, and/or of a process may include one or more of the aspects, features, concepts, examples, etc. described with reference to one or more of the examples discussed herein. Further, from figure to figure, the examples may incorporate the same or similarly named functions, steps, modules, etc. that may use the same, related, or unrelated reference numbers. The relevant features, elements, functions, operations, modules, etc. may be the same or similar functions or may be unrelated.

### Further Examples

In some aspects, the techniques described herein relate to a method including: receiving protocol data from a data source associated with an entity, the protocol data indicating at least one of a procedure, guideline, policy, or standard defined by the entity; receiving first user input data requesting generation of content relating to one or more parameters; processing the protocol data and the first user input data to generate a first data generation request that includes one or more constraints that are based on the protocol data and the first user input data; sending the first data generation request to a content generation component to generate content related to the first user input data; receiving machine-generated content from the content generation component; analyzing the machine-generated content to determine whether or not the machine-generated content aligns with the protocol data; in response to determining that the machine-generated content aligns with the protocol data: generating displayable content based on the machine-generated content; and providing the displayable content to a computing device; and in response to determining that the content does not align with the protocol data: generating a second data request, the second data request including one or more updated constraints; sending the second data request to the content generation component; receiving updated machine-generated content from the content generation component; and analyzing the updated machine-generated content.

In some aspects, the techniques described herein relate to a method, wherein the machine-generated content includes one or more terms and specification data, the specification data including at least one correct description of the one or more terms and at least one incorrect description of the one or more terms.

In some aspects, the techniques described herein relate to a method, further including: receiving second user input data requesting generation of content relating to the same one or more parameters; sending a second data request to the content generation component to generate content related to the second user input data; receiving additional machine-generated content from the content generation component, the additional machine-generated content being different than the machine-generated content; generating additional displayable content based on the additional machine-generated content; and providing the additional displayable content to the computing device.

In some aspects, the techniques described herein relate to a method, further including: receiving second user input data related to the displayable content; analyzing the second user input data to determine if the second user input data satisfies one or more criteria based on the protocol data or specification data; in response to determining that the second user input data satisfies the one or more criteria, provide an indication that the second user input data satisfies the one or more criteria.

In some aspects, the techniques described herein relate to a method, wherein the machine-generated content includes clinical data related to treating a medical condition.

In some aspects, the techniques described herein relate to a method, wherein the machine-generated content includes simulated laboratory results for a sample patient scenario.

In some aspects, the techniques described herein relate to a method, wherein the machine-generated content includes medication data related to a medication.

In some aspects, the techniques described herein relate to a method, further including: receiving at least one of patient data or sensor data generated by a medical device; determining a trend in a medical condition based on at least one of the patient data or sensor data; wherein generating the first data generation request is based on the trend.

In some aspects, the techniques described herein relate to a system including: control circuitry; and memory communicatively coupled to the control circuitry and storing executable instructions that, when executed by the control circuitry, cause the control circuitry to perform operations including: receiving protocol data from a data source associated with an entity, the protocol data indicating at least one of a procedure, guideline, policy, or standard defined by the entity defined by the entity; receiving first user input data requesting generation of content relating to one or more parameters; based on the protocol data, generating a first data generation request to generate content related to the first user input data; sending the first data generation request to a content generation component; receiving machine-generated content from the content generation component; generating displayable content based on the machine-generated content; and providing the displayable content to a computing device.

In some aspects, the techniques described herein relate to a system, wherein the operations further include: receiving a content item from a user; wherein the first data generation request includes the content item and a request to analyze the content item and generate content based on the analysis.

In some aspects, the techniques described herein relate to a system, wherein the operations further include: receiving patient data regarding medical conditions of patients; processing at least one of the patient data or the protocol data to generate analytics data indicating a trend in a medical condition; and wherein the first data generation request is generated based on the trend in the medical condition.

In some aspects, the techniques described herein relate to a system, wherein the analytics data indicates a trend in a medical condition for a particular geographic area.

In some aspects, the techniques described herein relate to a system, wherein the analytics data indicates a medical condition that is most frequently observed by a certain type of medical professional.

In some aspects, the techniques described herein relate to a system, wherein the displayable content indicates a medical condition that is most frequently diagnosed in a certain geographic area.

In some aspects, the techniques described herein relate to one or more non-transitory computer-readable media storing computer-executable instructions that, when executed, instruct one or more processors to perform operations including: receiving protocol data from a data source associated with an entity, the protocol data indicating at least one of a procedure, guideline, policy, or standard defined by the entity; receiving first user input data requesting generation of content relating to one or more parameters; based on the protocol data, generating a first data generation request to generate content related to the first user input data; sending the first data generation request to a content generation component; receiving machine-generated content from the content generation component; generating displayable content based on the machine-generated content; and providing the displayable content to a computing device.

In some aspects, the techniques described herein relate to one or more non-transitory computer-readable media, wherein the operations further include: receiving a content item from a user; wherein the first data generation request includes the content item and a request to analyze the content item and generate content based on the analysis.

In some aspects, the techniques described herein relate to one or more non-transitory computer-readable media, wherein the operations further include: receiving patient data regarding medical conditions of patients; processing at least one of the patient data or the protocol data to generate analytics data indicating a trend in a medical condition; and wherein the first data generation request is generated based on the trend in the medical condition.

In some aspects, the techniques described herein relate to one or more non-transitory computer-readable media, wherein the analytics data indicates a trends in a medical condition for a particular geographic area.

In some aspects, the techniques described herein relate to one or more non-transitory computer-readable media, wherein the analytics data indicates a medical condition that is most frequently observed by a certain type of medical professional.

In some aspects, the techniques described herein relate to one or more non-transitory computer-readable media, wherein the displayable content indicates a medical condition that is most frequently diagnosed in a certain geographic area.

In some aspects, the techniques described herein relate a method including: receiving protocol data; receiving user input data requesting generation of content relating to a topic; analyzing the protocol data to determine whether or not the protocol data includes content related to the topic; in response to determining that the protocol data includes content related to the topic, generate first displayable content based on the protocol data; in response to determining that the protocol data does not include content related to the topic, using an AI model to generate second displayable content; providing the first displayable content or the second displayable content via a user interface.

In some aspects, the techniques described herein relate a method including: receiving a content item; receiving user data indicating one or more parameters; analyzing the content item to identify a term that relates to the one or more parameters; using an AI model to generate correct data that relates to the term; using the AI model to generate inaccurate data that relates to the one or more terms; associating the inaccurate data and correct data with term data including the term; providing graphical interface data including graphical representations for the term, the correct data, and the inaccurate data; and enabling a user to select at least one of the graphical representations; receiving user input data including a selection of a first graphical representation of the graphical representations; processing the user input to determine whether or not the first graphical representation is associated with the correct data; providing an indication as to whether or not the first graphical representation represents a correct answer.

What is claimed is:

1. A method comprising:
receiving protocol data from a data source associated with an entity, the protocol data indicating at least one of a procedure, guideline, policy, or standard defined by the entity;
receiving first user input data requesting generation of content relating to one or more parameters;
processing the protocol data and the first user input data to generate a first data generation request that includes one or more constraints that are based on the protocol data and the first user input data;
sending the first data generation request to a content generation component to generate content related to the first user input data;
receiving machine-generated content from the content generation component;
analyzing the machine-generated content to determine whether or not the machine-generated content aligns with the protocol data;
in response to determining that the machine-generated content aligns with the protocol data:
generating displayable content based on the machine-generated content; and
providing the displayable content to a computing device; and
in response to determining that the content does not align with the protocol data:
generating a second data request, the second data request including one or more updated constraints;
sending the second data request to the content generation component;
receiving updated machine-generated content from the content generation component; and
analyzing the updated machine-generated content.

2. The method of claim 1, wherein the machine-generated content includes one or more terms and specification data, the specification data including at least one correct description of the one or more terms and at least one incorrect description of the one or more terms.

3. The method of claim 2, further comprising:
receiving second user input data requesting generation of content relating to the same one or more parameters;
sending a second data request to the content generation component to generate content related to the second user input data;
receiving additional machine-generated content from the content generation component, the additional machine-generated content being different than the machine-generated content;
generating additional displayable content based on the additional machine-generated content; and
providing the additional displayable content to the computing device.

4. The method of claim 2, further comprising:
receiving second user input data related to the displayable content;
analyzing the second user input data to determine if the second user input data satisfies one or more criteria based on the protocol data or specification data;
in response to determining that the second user input data satisfies the one or more criteria, provide an indication that the second user input data satisfies the one or more criteria.

5. The method of claim 1, wherein the machine-generated content includes clinical data related to treating a medical condition.

6. The method of claim 1, wherein the machine-generated content includes simulated laboratory results for a sample patient scenario.

7. The method of claim 1, wherein the machine-generated content includes medication data related to a medication.

8. The method of claim 1, further comprising:
receiving at least one of patient data or sensor data generated by a medical device;
determining a trend in a medical condition based on at least one of the patient data or sensor data;
wherein generating the first data generation request is based on the trend.

9. A system comprising:
control circuitry; and
memory communicatively coupled to the control circuitry and storing executable instructions that, when executed by the control circuitry, cause the control circuitry to perform operations comprising:
receiving protocol data from a data source associated with an entity, the protocol data indicating at least one of a procedure, guideline, policy, or standard defined by the entity defined by the entity;
receiving first user input data requesting generation of content relating to one or more parameters;
based on the protocol data, generating a first data generation request to generate content related to the first user input data;
sending the first data generation request to a content generation component;
receiving machine-generated content from the content generation component;
generating displayable content based on the machine-generated content; and
providing the displayable content to a computing device.

10. The system of claim 9, wherein the operations further comprise:
receiving a content item from a user;
wherein the first data generation request includes the content item and a request to analyze the content item and generate content based on the analysis.

11. The system of claim 9, wherein the operations further comprise:
receiving patient data regarding medical conditions of patients;
processing at least one of the patient data or the protocol data to generate analytics data indicating a trend in a medical condition; and
wherein the first data generation request is generated based on the trend in the medical condition.

12. The system of claim 11, wherein the analytics data indicates a trend in a medical condition for a particular geographic area.

13. The system of claim 11, wherein the analytics data indicates a medical condition that is most frequently observed by a certain type of medical professional.

14. The system of claim 11, wherein the displayable content indicates a medical condition that is most frequently diagnosed in a certain geographic area.

15. One or more non-transitory computer-readable media storing computer-executable instructions that, when executed, instruct one or more processors to perform operations comprising:
receiving protocol data from a data source associated with an entity, the protocol data indicating at least one of a procedure, guideline, policy, or standard defined by the entity;
receiving first user input data requesting generation of content relating to one or more parameters;
based on the protocol data, generating a first data generation request to generate content related to the first user input data;
sending the first data generation request to a content generation component;
receiving machine-generated content from the content generation component;
generating displayable content based on the machine-generated content; and
providing the displayable content to a computing device.

16. The one or more non-transitory computer-readable media of claim 15, wherein the operations further comprise:
receiving a content item from a user;
wherein the first data generation request includes the content item and a request to analyze the content item and generate content based on the analysis.

17. The one or more non-transitory computer-readable media of claim 15, wherein the operations further comprise:
receiving patient data regarding medical conditions of patients;
processing at least one of the patient data or the protocol data to generate analytics data indicating a trend in a medical condition; and
wherein the first data generation request is generated based on the trend in the medical condition.

18. The one or more non-transitory computer-readable media of claim 17, wherein the analytics data indicates a trend in a medical condition for a particular geographic area.

19. The one or more non-transitory computer-readable media of claim 17, wherein the analytics data indicates a medical condition that is most frequently observed by a certain type of medical professional.

20. The one or more non-transitory computer-readable media of claim 17, wherein the displayable content indicates a medical condition that is most frequently diagnosed in a certain geographic area.
